(19) United States
(12) Patent Application Publication
(10) Pub. No.: US 2007/0166269 A1
(43) Pub. Date: Jul. 19, 2007

(54) Cosmetic composition containing a statistical polymer with a linear chain of ethylenic nature

(75) Inventors: Guillaume Cassin, Villebon Sur Yvette (FR); Veronique Gourlaouen, Wissous (FR)

Correspondence Address: OLIFF & BERRIDGE, PLC, P.O. BOX 19928, ALEXANDRIA, VA, 22320, US

(73) Assignee: L'OREAL, PARIS (FR)

(21) Appl. No.: 11/605,955

(22) Filed: Nov. 30, 2006

Related U.S. Application Data
(60) Provisional application No. 60/749,636, filed on Dec. 13, 2005.

(30) Foreign Application Priority Data
Dec. 1, 2005 (FR) 05 53680

(51) Int. Cl. A61K 8/81

(52) U.S. Cl. 424/70.16

(57) ABSTRACT

The present invention relates to a cosmetic composition comprising, in a physiologically acceptable medium, at least one statistical copolymer with a linear main chain of ethylenic nature, in which the said copolymer has a molecular mass of between 15,000 and 600,000 g/mol, contains at least 70% of monomer units derived from monomers for which the homopolymers are hydrophobic and have a glass transition temperature of greater than 40° C., also contains at least one “monomer unit” derived from an at least partially neutralized ionic hydrophilic monomer, and has an overall glass transition temperature of greater than or equal to 45° C., and being in the form of a water-in-oil emulsion or a multiple emulsion.

This non provisional application claims the benefit of French Application No. 05 53680 filed on Dec. 1, 2005 and U.S. Provisional Application No. 60/749,636 filed on Dec. 13, 2005.

The present invention relates to an anti-wrinkle composition containing at least one statistical copolymer with a linear main chain of ethylenic nature characterized in that it is in the form of a water-in-oil emulsion or a multiple emulsion, and to the use of this copolymer as a tensioning agent in a cosmetic composition in the form of a water-in-oil emulsion or a multiple emulsion, intended especially for treating, reducing, effacing and/or smoothing out wrinkles and fine lines on human skin.

In the course of the ageing process, various signs appear on the skin, which are very characteristic of this ageing, which are reflected especially by a change in the structure and functions of the skin. The main clinical signs of ageing of the skin are especially the appearance of fine lines and deep wrinkles, which increase with age. Disorganization of the “grain” of the skin, i.e. the microrelief is less uniform and has an anisotropic nature, is in particular observed.

It is known practice to treat these signs of ageing by using cosmetic or dermatological compositions containing active agents capable of combating ageing, such as α-hydroxy acids, β-hydroxy acids and retinoids. These active agents act on wrinkles by removing the dead cells from the skin and by accelerating the process of cell renewal. However, these active agents have the drawback of being effective in treating wrinkles only after a certain period of application. Now, it is increasingly sought to obtain an immediate effect of the active agents used, rapidly leading to smoothing-out of wrinkles and fine lines and to the disappearance of fatigue marks.

The subject of the present invention is, precisely, the use, in a water-in-oil or multiple emulsion, of a particular copolymer that allows this effect to be obtained immediately.

It is known practice to use, in cosmetic compositions, synthetic polymers as tensioning agents.

Thus, WO 98/29092 discloses a composition with a tensioning effect comprising an aqueous dispersion of a polymeric system containing at least one polymer of synthetic origin with a molecular weight of greater than 670,000 g/mol, chosen from various types of polyurethanes, polyureas, acrylic polymers or copolymers and sulfonated isophthalic acid polymers, and mixtures thereof. The cosmetic feel of these compositions is, however, not always satisfactory.

EP 1 038 519 also discloses the use of certain specific silicone polymers for their role as agents with a tensioning effect, and WO 00/30595 discloses copolymers containing, by weight, from 20% to 90% of a vinyl lactam, from 1% to 55% of a polymerizable carboxylic acid and from 1% to 25% of a hydrophobic monomer such as a C₁₀ to C₂₄ alkyl acrylate or methacrylate, for providing soft gels that may be used in anti-wrinkle cosmetic compositions.

Finally, document FR 2 843 025 describes the use of interpenetrated polymer networks as agents for smoothing out wrinkles and fine lines and/or for retensioning the skin, and document FR 2 822 676 describes a film-forming cosmetic composition comprising, as film-forming agent, at least one acrylic copolymer in a high content, i.e. between 20% and 50% by weight relative to the weight of the composition.

Moreover, it is known that emulsions are commonly used in cosmetics. This is because they offer great flexibility in cosmetic formulations and fields of application. They may thus in particular include a wide variety of active ingredients of very different nature and may be in diverse forms such as more or less fluid or thick creams or gels.

Simple emulsions consist essentially of two immiscible phases, one fatty and the other aqueous, and of a surfactant whose role is to stabilize the dispersion of one of the phases, which is in the form of droplets dispersed within the continuous phase. Depending on the proportion of the two phases within the emulsion and on the nature of the surfactant, the emulsion is said to be of the oil-in-water (O/W) type when the aqueous phase is the continuous phase (direct emulsions), or of the water-in-oil (W/O) type when the fatty phase is the continuous phase (inverse emulsions).

Unfortunately, certain tensioning agents of synthetic polymer type have the major drawback of giving the compositions in emulsion form in which they are formulated insufficient stability. This instability may appear after a few days, or even a few weeks. It may be reflected by a drastic reduction in viscosity over time on storage, and/or by a “curdled” or even phase-separated appearance of the emulsion.

Such instability makes it difficult to use these tensioning copolymers of synthetic polymer type in cosmetic compositions in emulsion form, since these emulsions must show excellent stability throughout their commercial life.

There is thus a need to formulate cosmetic compositions in stable emulsion form comprising a tensioning agent of synthetic polymer type that offers an advantageous tensioning effect.

The inventors have discovered, unexpectedly, that statistical copolymers with a linear main chain of ethylenic type, characterized in that they:

(i) have a weight-average molecular mass of between 15,000 and 600,000 g/mol,

(ii) contain at least 70% of “monomer units” derived from monomers whose homopolymers are hydrophobic and have a glass transition temperature of greater than 40° C.,

(iii) also contain at least one “monomer unit” derived from an at least partially neutralized ionic hydrophilic monomer, and

(iv) have an overall glass transition temperature of greater than or equal to 45° C., show very advantageous tensioning effects while at the same time being able to be formulated in stable emulsion form. These compounds thus show very high rigidity after application to the skin and evaporation of the volatile materials, while at the same time offering an immediate, sufficient and long-lasting tensioning effect, without any risk to the consumer. These polymers also make it possible to prepare emulsions that are stable over time.

One subject of the present invention is thus a cosmetic composition comprising, in a physiologically acceptable medium, at least one statistical copolymer with a linear main chain of ethylenic nature, in which the said copolymer:

(i) has a weight-average molecular mass of between 15 000 and 600 000 g/mol,

(ii) contains at least 70% of “monomer units” derived from monomers whose homopolymers are hydrophobic and have a glass transition temperature of greater than 40° C.,

(iii) also contains at least one “monomer unit” derived from an at least partially neutralized ionic hydrophilic monomer, and

(iv) has an overall glass transition temperature of greater than or equal to 45° C., and being in the form of a water-in-oil emulsion or a multiple emulsion.

Another subject of the invention is the use as tensioning agent, in a cosmetic composition in the form of a water-in-oil emulsion or a multiple emulsion, of at least one statistical copolymer with a linear main chain of ethylenic nature, the said copolymer being as defined above.

Another subject of the invention is a cosmetic method for tensioning the skin comprising applying to said skin a cosmetic composition in the form of a water-in-oil or multiple emulsion containing a polymer as defined above.

In the context of this description and of the attached claims, the term “tensioning agent” means compounds capable of having an apparent tensioning effect, i.e. of smoothing out the skin and of immediately reducing, or even making disappear, the wrinkles and fine lines.

The composition in the form of a water-in-oil emulsion or a multiple emulsion used in the present invention contains, in addition to the abovementioned copolymer, a physiologically acceptable medium, i.e. a medium that is compatible with the skin and its integuments, mucous membranes and semi-mucous membranes.

Another subject of the invention is a cosmetic process for treating aged and for example wrinkled skin, comprising the application to the said skin of at least one composition according to the invention, in an amount that is effective for effacing the wrinkles by means of a tensioning effect.

One subject of the present invention is thus a cosmetic method for reducing or treating age signs comprising applying to the skin at least one composition comprising, in a physiologically acceptable medium, at least one statistical copolymer with a linear main chain of ethylenic nature, in which the said copolymer:

(i) has a weight-average molecular mass of between 15,000 and 600,000 g/mol,

(ii) contains at least 70% of monomer units derived from monomers whose homopolymers are hydrophobic and have a glass transition temperature of greater than 400 C,

(iii) also contains at least one “monomer unit” derived from an at least partially neutralized ionic hydrophilic monomer, and

(iv) as an overall glass transition temperature of greater than or equal to 45° C., and being in the form of a water-in-oil emulsion or a multiple emulsion, in an amount that is effective for smoothing out or effacing wrinkles and fine lines on human skin by means of a tensioning effect.

In the context of the present invention, the term “of ethylenic nature” qualifies polymers comprising a main chain comprising only monomer units, 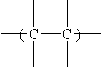
the side chains possibly consisting of carbon, oxygen, nitrogen, hydrogen, sulphur and/or phosphorus atoms.

It is noted in particular that the presence of silicon atoms in the side chain is excluded from the scope of the invention.

The term “monomer unit” denotes the largest constituent unit of the structure of a macromolecule formed from the same monomer molecule.

In the context of the present invention, the term “alkyl” means a linear or branched, saturated or unsaturated, cyclic or non-cyclic hydrocarbon-based chain. Among the alkyl groups that are suitable for use in the invention, mention may be made for example of methyl, ethyl, isopropyl, n-propyl, n-butyl, t-butyl, —CH₂-t-butyl, pentyl, n-hexyl, cyclopropyl, cyclopentyl, cyclohexyl, cyclohexylmethyl, heptyl, octyl, nonyl, decyl, norbomyl and adamantyl groups.

In the context of the present invention, the term “aryl” means a monocyclic or bicyclic system containing one or two aromatic nuclei. Among the aryl groups, mention may be made for example of phenyl, naphthyl, tetrahydronaphthyl and indanyl

The term “aralkyl” means an aryl group linked to an alkyl group, such as for example a benzyl group.

The term “heterocyclic group” means a 4- to 12-membered ring containing one or more identical or different heteroatoms chosen from O, N, S and P. The said heterocyclic group may or may not comprise double bonds. This term also comprises bicyclic groups in which a 3-, 4-, 5- or 6-membered heterocycle is fused to a phenyl group or to a cycloalkyl such as for example cyclohexane, or alternatively to another heterocycle.

Among these heterocyclic groups, mention may be made for example of indolyl, quinolyl, isoquinolyl, tetrahydroquinolyl, benzofuryl and benzothienyl. The term “heterocyclic group” especially covers pyrrolyl, pyrrolinyl, pyrrolidinyl, pyrazolyl, pyrazolinyl, pyrazolidinyl, imidazolyl, imidazolinyl, imidazolidinyl, pyridyl, piperidyl, pyrazinyl, piperazinyl, pyrimidinyl, pyridazinyl, oxazolyl, oxazolidinyl, isoxazolyl, isoxazolidinyl, morpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl and thienyl.

The term “heterocyclylalkyl” means a heterocyclic group linked to an alkyl group.

The halogen atoms comprise chlorine, bromine, iodine and fluorine.

Unless otherwise mentioned, the heteroatoms comprise oxygen, nitrogen, sulphur and phosphorus atoms.

For the purposes of the present invention, the term “emulsion” means a system containing at least two phases consisting of two or more immiscible or partially miscible liquids, one of which—which forms the dispersed phase—is dispersed in the other—which forms the continuous phase—in the form of fine droplets, the diameter of which does not exceed 5 microns.

The term “stable composition” denotes a composition that conserves well over time, for example after 1 month, or for example 2 months, at room temperature (25° C.), without phase separation or decantation.

The term “unstable composition” denotes a composition that shows decantation or phase separation after storage for at least 1 month at room temperature.

The stability of water-in-oil or multiple emulsions is for example reflected by good dispersion of the aqueous phase in the fatty phase and the absence of lumps of aqueous drops, promoting the appearance of regions of water of large diameter. The behaviour of the emulsion may be demonstrated by a simple test of dilution of the emulsion with oil, followed by its observation under a microscope. In the case of an unstable emulsion, the drops are aggregated and form lumps, which promotes sedimentation and the formation of water regions; the emulsion is then considered as unstable due to its heterogeneity created by the presence of its water regions. In the opposite case, good dispersion of the drops of aqueous phase and good stability of the emulsion, which is homogeneous, are observed.

The terms “between . . . and . . . ” and “ranging from . . . to” means that the limits are also included.

Copolymers

The copolymer present in the composition according to the present invention is a statistical copolymer with a linear main chain of ethylenic nature.

This copolymer may for example can be a statistical copolymer with a linear main chain of ethylenic nature:

(i) having a weight-average molecular mass of between 15,000 and 600,000 g/mol, and consisting of:

(ii) at least 70% by weight of “monomer units” derived from monomers for which the homopolymers are hydrophobic and have a glass transition temperature of greater than 40° C.,

(iii) at least one “monomer unit” derived from an at least partially neutralized ionic hydrophilic monomer, and

(iv) 0 to 25% by weight of “monomer units” derived from additional monomers for which the homopolymers have a glass transition temperature of less than 40° C., chosen from:
the acrylates of formula:
   CH₂═CHCOOR₁₂
   in which R₁₂ represents a linear or branched C₁ to C₁₂ alkyl group (with the exception of a tert-butyl group), in which is (are) optionally intercalated one or more identical or different heteroatoms, the said alkyl group also possibly being optionally substituted with one or more identical or different substituents chosen from hydroxyl groups and halogen atoms, and
the methacrylates of formula:
   CH₂═C(CH₃)COOR₁₄
   in which R₁₄ represents a linear C₄ to C₁₂ alkyl group or a branched C₅ to C₁₂ alkyl group, and

(v) having an overall glass transition temperature of greater than or equal to 45° C.

The composition may also comprise a mixture of such copolymers.

The copolymer that is useful in the context of the present invention may have a weight-average molecular mass of between 15,000 and 600,000 g/mol. The molecular mass may for example be between 20,000 and 200,000 g/mol and may for example range from 55,000 to 200,000 g/mol. The term “weight-average molecular mass” means the molecular mass Mw at the peak of the distribution curve.

Moreover, its overall glass transition temperature is greater than or equal to 45° C., for example ranging from 45° C. to 300° C.

The term “glass transition temperature”, the abbreviation of which is Tg, means the temperature below which the polymer is rigid. When the temperature increases, the polymer passes through a transition state that allows the macromolecular chains to slide relative to each other and the polymer softens.

The term “overall glass transition temperature” is used to indicate that the copolymer may include different monomers, the respective homopolymers of which may have different glass transition temperatures, and that it is the copolymer per se that has the said overall glass transition temperature.

Thus, in the context of the present invention, copolymers with an overall glass transition temperature of greater than 60° C., for example greater than 60° C. and less than 300° C., may be used.

In the context of the present invention, the protocol for measuring the glass transition temperatures of the copolymers or homopolymers formed by the monomers that are useful for preparing copolymers uses a characterization by DSC (Differential Scanning Calorimetry) and is detailed below:

The transitions of the film (glass transitions, melting, etc.) are studied by DSC on the basis of 2 cycles of heating/cooling at 10° C./minute between −140° C. and 130° C. (approximately 2 hours). The measurements are performed under a flush of nitrogen and using hermetic crucibles so as not to modify the composition of the film, by vaporization of the solvent, during the DSC study. The polymer film is prepared by drying the aqueous solution directly deposited (40 μl) in the thermal analysis crucibles. The drying of the solution takes place under controlled conditions over 48 hours at room temperature and at 50±5% relative humidity.
Apparatus: DSC 2920 from TA Instruments
Purge gas : Alphagaz 2 nitrogen at 50 ml/minute
Crucible: 50 μl crimped stainless-steel crucible from Perkin Elmer
Energy and temperature calibration: fusion of indium
Specimen: conditioned by drying (about 10 mg)
Heat treatments:
   1. CO: cooling from +25° C. to −140° C. at 10° C./minute
   2. COa: equilibration at −140° C.
   3. H1: heating from −140° C. to +130° C. at 10° C./minute
   4. C1: cooling from +130° C. to −140° C. at 10° C./minute
   5. C1a: equilibration at −140° C.
   6. H2: heating from −140° C. to +130° C. at 10° C./minute

Two samples are studied for each product.

The copolymer according to the invention may be in the form of an aqueous dispersion.

The structure of the copolymer is detailed in the description that follows.

The copolymer may comprise at least 70% by weight, relative to its total weight, of “monomer units” derived from monomers whose homopolymers have a glass transition temperature of greater than 40° C. These “monomer units” may be of the same nature or of different nature. In other words, the copolymer may comprise only one type of “monomer units” whose homopolymer has a glass transition temperature of greater than 40° C., or alternatively “monomer units” of different nature, it being understood that the corresponding monomers are all in accordance with the requirement recalled above concerning the glass transition temperature.

In particular, the copolymer according to the invention may comprise more than 72%, for example more than 75% and for example more than 80% by weight, and for example up to 95% by weight, relative to its total weight, of “monomer units” derived from monomers whose homopolymers have a glass transition temperature of greater than 40° C.

The copolymer may also comprise at least one “monomer unit” derived from an ionic hydrophilic monomer, and also from 0 to 25% by weight of “monomer units” derived from additional monomers whose homopolymers have a glass transition temperature of less than 40° C.

Hydrophobic Monomers Whose Homopolymers have a Lass Transition Temperature of Greater than 40° C.

For the purposes of the present invention, the term “hydrophobic monomer” means a monomer whose homopolymer is insoluble in water at a concentration of greater than 5% by weight, at 25° C., and which does not form either in water, under these conditions, a stable dispersion or suspension of fine, generally spherical, particles with a mean particle size of less than 1 μm, and more for example between 5 and 400 nm, or for example between 10 and 250 nm, as measured by light scattering.

Among the monomers from which the hydrophobic “monomer units” mentioned above are derived, those whose homopolymers have a glass transition temperature of greater than or equal to 60° C. and for example less than or equal to 300° C. may for example be used.

The monomers that are useful for the preparation of the copolymers included in the compositions according to the present invention and whose homopolymers have glass transition temperatures of greater than 40° C. may for example be chosen from vinyl compounds, acrylates, methacrylates and (meth)acrylamides, and for example from the following monomers:
the vinyl compounds of formula:
   CH₂═CHR₁,

in which R₁ is a group: 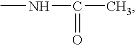
a group

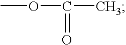

a C₃ to C₈ cycloalkyl group; a C₆ to C₂₀ aryl group; a C₇ to C₃₀ aralkyl group (C₁ to C₄ alkyl group); a heterocyclic group; a heterocyclylalkyl group (C₁ to C₄ alkyl) such as a furfuryl group; the said cycloalkyl, aryl, aralkyl, heterocyclic or heterocyclylalkyl groups possibly being substituted with one or more identical or different substituents chosen from hydroxyl groups, halogen atoms and linear or branched C₁ to C₄ alkyl groups in which is (are) optionally intercalated one or more identical or different heteroatoms, and the said alkyl groups also possibly being substituted with one or more substituents chosen from hydroxyl groups and halogen atoms.

According to one exemplary embodiment, R₁ is a group 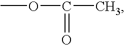
a C₃ to C₈ cycloalkyl group or a C₆ to C₂₀ aryl group.

According to another exemplary embodiment, the vinyl monomers whose homopolymers have a glass transition temperature of greater than 40° C. may be vinylcyclohexane, styrene and vinyl acetate.
The acrylates of formula:
   CH₂═CHCOOR₂

in which R₂ is a tert-butyl group; a C₃ to C₈ cycloalkyl group optionally bridged with a C₁ to C₄ alkylene group optionally substituted with one or more C₁ to C₄ alkyl groups; a C₆ to C₂₀ aryl group; a C₇ to C₃₀ aralkyl group having a C₁ to C₄ alkyl group; a heterocyclic group; a heterocyclylalkyl group (C₁ to C₄ alkyl); the said cycloalkyl, aryl, aralkyl, heterocyclic or heterocyclylalkyl groups possibly being substituted with one or more identical or different substituents chosen from hydroxyl groups, halogen atoms and linear or branched C₁ to C₄ alkyl groups in which is (are) optionally intercalated one or more identical or different heteroatoms, the said alkyl groups also possibly being substituted with one or more identical or different substituents chosen from hydroxyl groups and halogen atoms.

According to one exemplary embodiment, R₂ is a tert-butyl group, a C₃ to C₈ cycloalkyl group optionally bridged with a C₁ to C₄ alkylene group, or a C₇ to C₃₀ aralkyl group (C₁ to C₄ alkyl group).

According to another exemplary embodiment, the acrylates whose homopolymers have a glass transition temperature of greater than 40° C. may be benzyl acrylate, cyclohexyl acrylate, tert-butyl acrylate, isobomyl acrylate and norbomyl acrylate.
The methacrylates of formula:
   CH₂═C(CH₃)COOR₃

in which R₃ is an isobutyl or tert-butyl or a linear or branched C₁ to C₃ alkyl group, such as a methyl, ethyl, propyl or isobutyl group, these alkyl groups also possibly being substituted with one or more identical or different substituents chosen from hydroxyl groups and halogen atoms; a C₃ to C₈ cycloalkyl group optionally bridged with a C₁ to C₄ alkylene group optionally substituted with one or more C₁ to C₄ alkyl groups; a C₆ to C₂₀ aryl group; a C₇ to C₃₀ aralkyl group having a C₁ to C₄ alkyl group; a heterocyclic group; a heterocyclylalkyl group (C₁ to C₄ alkyl); the said cycloalkyl, aryl, aralkyl, heterocyclic or heterocyclylalkyl groups possibly being substituted with one or more identical or different substituents chosen from hydroxyl groups, halogen atoms and linear or branched C₁ to C₄ alkyl groups in which is (are) optionally intercalated one or more identical or different heteroatoms, the said alkyl groups also possibly being substituted with one or more identical or different substituents chosen from hydroxyl groups and halogen atoms.

According to one exemplary embodiment, R₃ is an isobutyl or tert-butyl group, a linear or branched C₁ to C₃ alkyl group, a C₃ to C₈ cycloalkyl group optionally bridged with a C₁ to C₄ alkylene group, or a C₇ to C₃₀ aralkyl group (C₁ to C₄ alkyl group).

According to another exemplary embodiment, the methacrylates whose homopolymers have a glass transition temperature of greater than 40° C. may be methyl, ethyl, isobutyl, cyclohexyl, benzyl, tert-butyl, isobomyl and norbornyl methacrylate.
The (meth)acrylamides of formula:

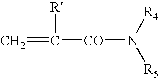

in which R′ denotes H or —CH₃, and in which R₄ and R₅, which may be identical or different, each represent a hydrogen atom or a linear or branched linear C₄ to C₁₂ alkyl group, it being understood that R₄ and R₅ cannot simultaneously represent a hydrogen atom.

According to one exemplary embodiment, the (meth)acrylamide monomers whose homopolymers have a glass transition temperature of greater than 40° C. may be N-butylacrylamide, N-t-butylacrylamide and N,N-dibutylacrylamide.

According to one exemplary embodiment of the invention, the monomers whose homopolymers have a glass transition temperature of greater than 40° C. may be elected from the group consisting of styrene, benzyl acrylate, C₃ to C₈ cycloalkyl acrylate ptionally bridged with a C₁ to C₄ alkylene group, tert-butyl acrylate, C₁ to C₃ alkyl ethacrylate, tert-butyl methacrylate, benzyl methacrylate and C₃ to C₈ cycloalkyl ethacrylate optionally bridged with a C₁ to C₄ alkylene group.

The monomers whose homopolymers have a glass transition temperature of greater than 40° C. may be selected from the group consisting of benzyl acrylate, cyclohexyl acrylate, tert-butyl acrylate, isobomyl acrylate, norbornyl acrylate, methyl, ethyl, isobutyl, cyclohexyl, benzyl, tert-butyl, isobornyl or norbornyl methacrylate and styrene. They may for example be selected from the group consisting of methyl methacrylate and cyclohexyl methacrylate.

Ionic Hydrophilic Monomer

The copolymer present in the composition according to the present invention also comprises at least one “monomer unit” derived from an at least partially neutralized ionic hydrophilic monomer.

This ionic hydrophilic monomer may be for example selected from the group consisting of anionic hydrophilic monomers, cationic hydrophilic monomers and amphoteric monomers, and mixtures thereof.

The copolymer may for example contain between 5% and 30% by weight of “monomer units” derived from ionic hydrophilic monomers, relative to its total weight. The copolymer may for example contain between 5% and 25% by weight, for example between 5% and 20% by weight and for example between 5% and 18% by weight of “monomer units” derived from ionic hydrophilic monomers, relative to its total weight. The copolymer may contain, for example, between 10% and 25% by weight, for example between 10% and 20% by weight and for example between 10% and 18% by weight of “monomer units” derived from ionic hydrophilic monomers relative to its total weight.

According to one exemplary embodiment, the copolymer may contain between 5% and 25% by weight of “monomer units” derived from ionic hydrophilic monomers, it being understood that when the copolymer contains one or more additional monomer(s) whose respective homopolymer(s) has (have) a glass transition temperature of less than 40° C., at least one of these additional monomer(s) being ethylhexyl acrylate, then the copolymer contains between 5% and 18% by weight of “monomer units” derived from ionic hydrophilic monomers.

For the purposes of the present invention, the term “partially neutralized” means a non-zero degree of neutralization, for examplegreater than or equal to 50%.

According to one exemplary embodiment, all the “monomer units” may have a degree of neutralization of greater than or equal to 50%, for example greater than or equal to 70% and for example of at least 90%, or for example of 100%.

The degree of neutralization may be defined as being the ratio of the number of neutralized ionic functions to the initial number of ionized functions, i.e. before neutralization.

According to one exemplary embodiment, the degree of neutralization of all the “monomer units” may be adjusted so as to allow dispersion in water of the copolymers according to the invention.

Among the anionic hydrophilic monomers are monomers of formula (I):
CH₂═CR₆(Z)ₙ(R₇)ₘY (I)

in which Z has one of the following meanings: C(═O)O, C(═O)NH, O, O(C═O) (acetates),

n is equal to 0 or 1, for example equal to 0,

m is equal to 0 or 1, for example equal to 0,

R₆ is a hydrogen atom, a CH₃ group or a (C₂-C₃₀)alkyl group,

R₇ is selected from the group consisting of linear, saturated or unsaturated and/or branched and/or cyclic (aromatic or non-aromatic) C₁ to C₃₀ alkylene groups, optionally including one or more heteroatoms, and

Y is selected from the following groups: —COOH, —SO₃H, —OSO₃H, —OP(OH)₂ and —OPO(OH)₂.

According to one exemplary embodiment, R₆ may be a hydrogen atom or a CH₃ or C₂H₅ group, for example CH₃.

R₇ may beselected, for example, from the group consisting of C₁ to C₃₀ alkylene, phenylene, benzylene, —(CH₂—CH═CH)— and —(CHOH)— groups. According to one exemplary embodiment, R₇ may be chosen from linear, branched or cyclic, C₁ to C₆ alkylene groups and phenylene and benzylene groups.

According to one exemplary embodiment, R₆ may be a hydrogen atom or a CH₃ or a C₂H₅ group, for example a CH₃ group, and R₇ may be chosen from C₁ to C₃₀ alkylene, phenylene, benzylene, (CH₂—CH═CH)— and (CHOH) groups.

Among the anionic hydrophilic monomers, mention may be made for example of:
ethylenically unsaturated monomers comprising at least one carboxylic acid (COOH), phosphonic acid (PO₃H₂) or sulfonic acid (SO₃H) finction, for instance acrylic acid, methacrylic acid, 2-carboxyethyl (meth)acrylate, vinylbenzoic acid, acrylamido-glycolic acid CH₂═CHCONHCH(OH)CO₂H, maleic acid, acrylamidopropanesulfonic acid, acrylamido-2-methylpropanesulfonic acid, styrenesulfonic acid, vinylsulfonic acid, 3-propylsulfonic acid methacrylate or acrylate (CH₂═C(CH₃)CO₂(CH₂)₃SO₃H), 2-ethyl-sulfonic acid methacrylate or acrylate (CH₂═C(CH₃)CO₂(CH₂)₂SO₃H) and methyl vinyl sulfone, vinylphosphonic acid (CH₂═CH—PO(OH)₂) and 2-ethylphosphonic acid methacrylate (CH₂═C(CH₃)COOCH₂CH₂OP(O)(OH)₂),
carboxylic anhydrides bearing a vinyl bond, such as for example maleic anhydride,
diacids such as crotonic acid, itaconic acid, fumaric acid or maleic acid,
and mixtures thereof.

According to one exemplary embodiment, the anionic hydrophilic monomer may be (meth)acrylic acid.

Neutralization of the anionic groups may be performed with a mineral base, such as LiOH, NaOH, KOH, Ca(OH)₂, NH₄OH or Zn(OH)₂; or with an organic base such as a primary, secondary or tertiary alkylamine, for example triethylamine or butylamine. This primary, secondary or tertiary alkylamine may comprise one or more nitrogen and/or oxygen atoms and may thus comprise, for example, one or more alcohol finctions; mention may be made for example of 2-amino-2-methylpropanol, triethanolamine and 2-dimethylaminopropanol. Mention may also be made of lysine or 3-(dimethylamino)propylamine.

Among the cationic hydrophilic monomers that are included are the monomers of formula (II):
CH₂═CR₈(Z)ₙ(R₉)ₘX (II)

in which:
Z takes one of the following meanings: C(═O)O, C(═O)NH, O, O(C═O),
n is equal to 0 or 1,
m is equal to 0 or 1,
R₈ is a hydrogen atom, a CH₃ group or a (C₂ to C₃₀)alkyl group,
R₉ is chosen from linear, saturated or unsaturated and/or branched and/or cyclic, aromatic or non-aromatic C₁ to C₃₀ alkylene groups, optionally including one or more heteroatoms,
X is a group defined by N—R₁₀R₁₁, or alternatively X constitutes an aromatic or non-aromatic ring, comprising a cationizable tertiary amine group, included in the ring or as a substituent, or may represent an aromatic or non-aromatic heterocycle containing a cationizable tertiary nitrogen included in the ring or as a substituent, or alternatively X takes one of the following meanings: guanidino, amidino or phosphino, and
R₁₀ and R₁₁, are chosen, independently, from a hydrogen atom and linear and/or branched and/or cyclic, aromatic or non-aromatic C₁ to C₆ alkyl groups, optionally comprising heteroatoms, or alternatively R₁₀ and R₁₁ form, with the nitrogen to which they are attached, a fused or non-fused C₄ to C₃₀ heterocycle, optionally substituted with one or more identical or different radicals chosen from C₁ to C₄ alkyl groups, a hydroxyl group, a C₁ to C₄ alkoxy group and a halogen atom.

According to one exemplary embodiment, R₉ may be chosen from C₁ to C₃₀ alkyl, phenylene, benzylene, —(CH₂—CH═CH)— and —(CHOH)— groups.

According to one exemplary embodiment, R₉ may be chosen from linear, branched or cyclic C₁ to C₆ alkylene groups, and phenylene and benzylene groups.

According to one exemplary embodiment, R₁₀ and R₁₁, may be chosen from a hydrogen atom and CH₃, C₂H₅, C₃H₇ and C₄H₉ groups.

Examples of heterocycles that are suitable for the meaning of X in its second alternative are pyridines, indolyl, isoindolinyl, imidazolyl, imidazolinyl, piperidyl, pyrazolinyl, pyrazolyl, quinoline, pyrazolinyl, pyridinyl, piperazinyl, pyrrolidinyl, quinidinyl, thiazolinyl, morpholine, and mixtures thereof.

Examples of cationic hydrophilic monomers that may be mentioned include ethylenically unsaturated monomers comprising at least one primary, secondary or tertiary amine function. Mention may be made for example of:
allylamine, allylpyridine;
aminoalkyl (meth)acrylates, such as for example [N,N-di(C₁-C₄)alkylamino](C₁-C₆)alkyl (meth)acrylates or [N—(C₁-C₄)alkylamino](C₁-C₆)alkyl (meth)acrylates and for example N,N dimethylaminoethyl (meth)acrylate, N,N-diethylaminoethyl (meth)acrylate, 2-aminoethyl (meth)acrylate and 2-(N-tert-butylamino)ethyl (meth)acrylate;
aminoalkyl(meth)acrylamides, such as for example (meth)acrylamides of [N,N-di(C₁-C₄)alkylamino](C₁-C₆)alkyl N,N-di(C₁-C₄)alkylamino)alkyl or [N—(C₁-C₄)alkylamino](C₁-C₆)alkyl(meth)acrylamides, and for example N,N-dimethylaminopropyl(meth)acrylamide, N,N-dimethylaminoethyl(meth)acrylamide and 3-aminopropyl(meth)acrylamide;
vinylamine, 2-(diethylamino)ethylstyrene;
N-vinylimidazole, N-vinyl-2-methylimidazole, N-vinylcarbazole;
and also mixtures thereof, and quaternized forms thereof

Salts of mineral acids, such as sulphuric acid, hydrochloric acid, hydrobromic acid, hydriodic acid, phosphoric acid and boric acid, may be used to neutralize the cationic monomers.

Salts of organic acids, which may comprise one or more carboxylic, sulfonic or phosphonic acid groups, may also be mentioned. These may be linear, branched or cyclic aliphatic acids or alternatively aromatic acids. These acids may also comprise one or more heteroatoms chosen from O and N, for example in the form of hydroxyl groups. Mention may be made for example of propionic acid, acetic acid, terephthalic acid, citric acid and tartaric acid.

The tertiary amine groups may be quaternized:
with compounds containing labile halogen, especially alkyl halides such as for example C₁ to C₁₂ alkyl chlorides or bromides, for example methyl bromide or ethyl chloride,
compounds containing labile halogen, comprising carboxylic or sulfonic acid functions (optionally salified). Thus, amphoteric hydrophilic monomers (also known as betaines) are obtained.

The quaternizing agents may be, for example, sodium chloroacetate or cyclic sulfones, for example propane sulfone.

This quaternization and (or) salification reaction may take place on the already-synthesized polymer or on the starting monomer before performing the polymerization.

Examples of amphoteric monomers that may be mentioned include ethylenic carboxybetains or sulfobetains such as for example N,N-dimethyl-N-(2-methacryloyloxyethyl)-N-(3-sulfopropyl)ammonium betaine; N,N-dimethyl-N-(3-methacrylamidopropyl)-N-(3-sulfopropyl)ammonium betaine and 1-(3-sulfopropyl)-2-vinylpyridinium betaine.

The cationic hydrophilic monomer is may for example be N,N′-dimethylaminoethyl (meth)acrylate.

The hydrophilic monomer is may for example be (meth)acrylic acid.

Preferred Copolymers

According to one exemplary embodiment, the copolymer may be selected from the group consisting of:
methyl methacrylate/methacrylic acid copolymers; methyl methacrylate/acrylic acid copolymers, the said copolymers containing between 70% and 90% by weight of methyl methacrylate;
ethyl methacrylate/methacrylic acid copolymers; ethyl methacrylate/acrylic acid copolymers, the said copolymers containing between 70% and 90% by weight of ethyl methacrylate;
isobutyl methacrylate/methacrylic acid copolymers; isobutyl methacrylate/acrylic acid copolymers, the said copolymers containing between 70% and 90% by weight of isobutyl methacrylate;
benzyl methacrylate/methacrylic acid copolymers; benzyl methacrylate/acrylic acid copolymers, the said copolymers containing between 70% and 90% by weight of benzyl methacrylate;
benzyl acrylate/methacrylic acid copolymers; benzyl acrylate/acrylic acid copolymers, the said copolymers containing between 70% and 90% by weight of benzyl acrylate;
cyclohexyl methacrylate/methacrylic acid copolymers; cyclohexyl methacrylate/acrylic acid copolymers, the said copolymers containing between 70% and 90% by weight of cyclohexyl methacrylate;
cyclohexyl acrylate/methacrylic acid copolymers; cyclohexyl acrylate/acrylic acid copolymers, the said copolymers containing between 70% and 90% by weight of cyclohexyl acrylate;
tert-butyl methacrylate/methacrylic acid copolymers; tert-butyl methacrylate/acrylic acid copolymers, the said copolymers containing between 70% and 90% by weight of tert-butyl methacrylate;
tert-butyl acrylate/methacrylic acid copolymers; tert-butyl acrylate/acrylic acid copolymers, the said copolymers containing between 70% and 90% by weight of tert-butyl acrylate;
isobornyl methacrylate/methacrylic acid copolymers; isobornyl methacrylate/acrylic acid copolymers, the said copolymers containing between 70% and 90% by weight of isobornyl methacrylate;
isobornyl acrylate/methacrylic acid copolymers; isobornyl acrylate/acrylic acid copolymers, the said copolymers containing between 70% and 90% by weight of isobornyl acrylate;
norbornyl methacrylate/methacrylic acid copolymers; norbornyl methacrylate/acrylic acid copolymers, the said copolymers containing between 70% and 90% by weight of norbornyl methacrylate;
norbornyl acrylate/methacrylic acid copolymers; norbornyl acrylate/acrylic acid copolymers, the said copolymers containing between 70% and 90% by weight of norbornyl acrylate; and
styrene/methacrylic acid copolymers; styrene/acrylic acid copolymers, the said copolymers containing between 70% and 90% by weight of styrene.

Monomer with a Tg<40° C.

According to one exemplary embodiment, the copolymers also comprising at least one “monomer unit” derived from a monomer whose corresponding homopolymer has a glass transition temperature of less than 40° C. (and for example greater than −100° C.) may also form part of the invention provided that the overall glass transition temperature of the said copolymer remains very much higher than or equal to 45° C.

According to another exemplary embodiment, the additional monomers whose homopolymers have a glass transition temperature of less than 40° C., which are known as “monomers with a Tg<40° C.”, may be different from the hydrophobic monomers and the ionic hydrophilic monomers described above.

The content of monomers whose homopolymer has a glass transition temperature of less than 40° C. is adjusted such that the tensioning effect of the copolymer is not affected.

The content in the copolymer may for example be less than or equal to 25% by weight (for example from 0 to 25% by weight and for example from 5% to 25% by weight) and for example less than or equal to 10% by weight (for example from 0 to 10% by weight and for example from 5% to 10% by weight), relative to the total weight of the copolymer.

The monomers whose homopolymers have glass transition temperatures of less than 40° C. may be chosen from the following monomers:
C₂ to C₁₀ ethylenic hydrocarbons, such as for example ethylene, isoprene and butadiene,
the acrylates of formula:
   CH₂═CHCOOR₁₂

in which R₁₂ represents:
a linear or branched C₁ to C₁₂ alkyl group (with the exception of a tert-butyl group), in which is (are) optionally intercalated one or more identical or different heteroatoms, the said alkyl group also possibly being substituted with one or more identical or different substituents chosen from hydroxyl groups and halogen atoms (especially fluorine), for example a linear or branched C₁ to C₁₂ alkyl group (with the exception of a tert-butyl group); examples of groups R₁₂ are methyl, ethyl, propyl, butyl, isobutyl, hexyl, ethylhexyl, octyl, lauryl, isooctyl, isodecyl, hydroxyethyl, hydroxypropyl, methoxyethyl, ethoxyethyl and methoxypropyl groups;
a C₁ to C₁₂ alkyl-POE (polyoxyethylene) group, with repetition of the oxyethylene unit from 5 to 30 times, for example methoxy POE, R12 then possibly being represented by R13—(OC2H4)n—, in which R13 is a C1 to C12 alkyl group and n is an integer ranging from 5 to 30; or
a polyoxyethylene group of structure —(CH2CH2O)n—H in which n is an integer ranging from 5 to 30.
the methacrylates of formula:
   CH₂═C(CH₃)COOR₁₄

in which R₁₄ represents:
a linear C4 to C12 alkyl group or a branched C5 to C12 alkyl group (with the exception of cycloalkyl, aryl, aralkyl, heterocycloalkyl and heteroaralkyl groups), in which is (are) optionally intercalated one or more identical or different heteroatoms, the said alkyl group also possibly being substituted with one or more identical or different substituents chosen from hydroxyl groups and halogen atoms; and for example a linear C4 to C12 alkyl group or a branched C5 to C12 alkyl group, which is unsubstituted; examples of groups R14 are hexyl, ethylhexyl, octyl, lauryl, isooctyl, isodecyl, dodecyl, methoxyethyl, methoxypropyl and ethoxyethyl groups,
a C₁ to C12 alkyl-POE (polyoxyethylene) group, with repetition of the oxyethylene unit from 5 to 30 times, for example methoxy POE, R14 then possibly being represented by R13—(OC2H4)n—, in which R13 is a C₁ to C12 alkyl group and n is an integer ranging from 5 to 30; or
a polyoxyethylene group of structure —(CH2CH2O)n—H in which n is an integer ranging from 5 to 30.

According to one exemplary embodiment, the monomers may be: methyl acrylate, ethyl acrylate, propyl acrylate, n-butyl acrylate, 2-ethylhexyl acrylate, isobutyl acrylate, methoxyethyl acrylate, ethoxyethyl (meth)acrylate, n-hexyl (meth)acrylate.
The vinyl esters of formula:
   R₁₅COOCH═CH₂

in which R₁₅ represents a linear or branched C₂ to C₁₂ alkyl group.

Examples of such vinyl esters are: vinyl propionate, vinyl butyrate, vinyl ethylhexanoate, vinyl neononanoate and vinyl neododecanoate.
Ethers of vinyl alcohol and of a C₁ to C₁₂ alcohol, such as for example methyl vinyl ether and ethyl vinyl ether.

The additional monomer may for example be preferably chosen from the acrylates and methacrylates described above, and for example from C₁-C₁₂ alkyl acrylates and C₄-C₁₂ alkyl methacrylates.

According to one exemplary embodiment, the preferred additional monomers may be:
the acrylates of formula:
   CH₂═CHCOOR₁₂

in which R₁₂ represents a linear or branched C₁ to C₁₂ alkyl group (with the exception of a tert-butyl group), in which is (are) optionally intercalated one or more identical or different heteroatoms, the said alkyl group also possibly being substituted with one or more identical or different substituents chosen from hydroxyl groups and halogen atoms, and
the methacrylates of formula:
   CH₂═C(CH₃)COOR₁₄

in which R₁₄ represents a linear C₄ to C₁₂ alkyl group or a branched C₅ to C₁₂ alkyl group.

According to one exemplary embodiment , the additional monomer may be one of the monomers detailed above, with the exclusion of ethylhexyl acrylate.

Composition

The content, expressed as dry matter, of copolymer(s) present in a composition according to the present invention may range from 0.1% to 15%, for example from 1% to 10% and for example from 1% to 5% by weight relative to the total weight of the composition.

The cosmetic composition according to the present invention is in the form of a water-in-oil emulsion or a multiple emulsion.

Moreover, in the context of the present invention, the term “multiple emulsion” covers water-in-oil-in-water, oil-in-water-in-oil and water-in-silicone oil-in-water emulsions, the water-in-oil-in-water emulsion being one exemplary embodiment.

Water-in-Oil (W/O) Inverse Emulsions

According to one exemplary embodiment, the composition according to the invention may be in the form of a water-in-oil W/O emulsion.

The composition according to the invention may be prepared according to any method known to those skilled in the art for preparing water-in-oil W/O emulsions. It may for example be a water-in-oil W/O emulsion comprising a gelled or non-gelled inner aqueous phase and a fatty phase comprising at least one oil and at least one surfactant.

Aqueous phase

The aqueous phase forms the dispersed phase of the W/O emulsion according to the invention.

The aqueous phase comprises water and may consist essentially of water.

It may also comprise a mixture of water and of water-miscible organic solvent (miscibility in water of greater than 50% by weight at 25° C.), for instance lower monoalcohols containing from 1 to 5 carbon atoms such as for example ethanol or isopropanol, glycols containing from 2 to 8 carbon atoms such as for example propylene glycol, ethylene glycol, 1,3-butylene glycol or dipropylene glycol, C₃-C₁₄ ketones and C₂-C₄ aldehydes.

The aqueous phase (water and optionally the water-miscible organic solvent) may be present in a content ranging from 1% to 95% by weight, for example ranging from 5% to 80% by weight and for example ranging from 15% to 60% by weight relative to the total weight of the composition in the form of a water-in-oil emulsion.

The inner aqueous phase may be gelled or non-gelled. According to one exemplary embodiment, the inner aqueous phase is non-gelled.

Fatty phase

The fatty phase forms the continuous phase of the W/O emulsion according to the invention.

It comprises at least one fatty substance that is liquid at room temperature (25° C.) such as for example an oil, where appropriate combined with a fatty substance that is solid at room temperature such as for example waxes, pasty fatty substances and gums, and mixtures thereof. The fatty phase may also contain lipophilic organic solvents.

The fatty phase content may range from 5% to 80% by weight and for example from 5% to 50% by weight relative to the total weight of the composition in the form of a water-in-oil emulsion.

Oil

The fatty phase of the composition according to the invention may for example comprise, as liquid fatty substance, at least one volatile or non-volatile oil or a mixture thereof.

According to one exemplary embodiment, the fatty phase of a composition in accordance with the invention may comprise at least one volatile or non-volatile silicone oil.

For the purposes of the invention, the term “volatile oil” means any oil capable of evaporating on contact with the skin in less than one hour, at room temperature and atmospheric pressure. The volatile oils of the invention are volatile cosmetic oils, which are liquid at room temperature, with a non-zero vapour pressure, at room temperature and atmospheric pressure, ranging in particular from 0.01 to 300 mm Hg (1.33 Pa to 40 000 Pa) and for example greater than 0.3 mm Hg (30 Pa).

The term “non-volatile oil” means an oil that remains on the skin at room temperature and atmospheric pressure for at least several hours and that has for example a vapour pressure of less than 0.01 mm Hg (1.33 Pa).

These volatile or non-volatile oils may be hydrocarbon-based oils or silicone oils, or mixtures thereof.

The term “hydrocarbon-based oil” means an oil mainly containing hydrogen and carbon atoms and possibly oxygen, nitrogen, sulfur or phosphorus atoms, and containing no silicon or fluorine atoms. It may contain alcohol, ester, carboxylic acid, amine and/or amide groups.

The volatile hydrocarbon-based oils may be chosen from hydrocarbon-based oils containing from 8 to 16 carbon atoms, and for example branched C₈-C₁₆ alkanes, for instance C₈-C₁₆ isoalkanes of petroleum origin (also known as isoparaffins), for instance isododecane (also known as 2,2,4,4,6-pentamethylheptane), isodecane, isohexadecane and, for example, the oils sold under the trade names Isopar® and Permethyl®, and branched C₈-C₁₆ esters such as for example isohexyl neopentanoate, and mixtures thereof.

Other volatile hydrocarbon-based oils, for instance petroleum distillates, for example those sold under the name Shell Solt® by the company Shell, may also be used. 30 Volatile oils that may also be used include volatile silicones, for instance volatile linear or cyclic silicone oils, for example those with a viscosity ≦8 centistokes (8×10⁻⁶ m²/s) and for example containing from 2 to 7 silicon atoms, these silicones optionally comprising alkyl or alkoxy groups containing from 1 to 10 carbon atoms. As volatile silicone oils that may be used in the invention, mention may be made for example of octamethylcyclotetrasiloxane, decamethylcyclopentasiloxane, dodecamethylcyclohexa-siloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

The non-volatile oils may be chosen for examplefrom non-volatile fluoro and/or silicone hydrocarbon-based oils.

Non-volatile hydrocarbon-based oils that may be for example mentioned include:
hydrocarbon-based oils of animal origin, for instance mink oil, turtle oil or perhydrosqualene, - hydrocarbon-based oils of plant origin, such as for example triglycerides consisting of fatty acid esters of glycerol, the fatty acids of which may have chain lengths ranging from C₄ to C₂₄, these chains possibly being linear or branched, and saturated or unsaturated; these oils are for example wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, sesame oil, marrow oil, rapeseed oil, blackcurrant pip oil, evening primrose oil, millet oil, barley oil, quinoa oil, rye oil, safflower oil, candlenut oil, passion flower oil or musk rose oil; shea butter; or caprylic/capric acid triglycerides, for instance those sold by the company Stearineries Dubois or those sold under the names Miglyol 810, 812 and 818® by the company Dynamit Nobel;
synthetic ethers containing from 10 to 40 carbon atoms;
linear or branched hydrocarbons of mineral or synthetic origin, such as for example liquid paraffin or derivatives thereof, petroleum jelly, polydecenes, hydrogenated polyisobutene such as for example Parléam® sold by the company Nippon Oil Fats, and squalane, and mixtures thereof;
synthetic esters, for instance oils of formula R₁COOR₂ in which R₁ represents a linear or branched fatty acid residue containing from 1 to 40 carbon atoms and R₂ represents a hydrocarbon-based chain, which is for examplebranched, containing from 1 to 40 carbon atoms, on condition that R₁+R₂ is ≧10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, and heptanoates, octanoates, decanoates or ricinoleates of alcohols or of polyalcohols, for instance propylene glycol dioctanoate; hydroxylated esters, for instance isostearyl lactate or diisostearyl malate; polyol esters and pentaerythritol esters;
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
C₈-C₂₆ higher fatty acids such as for example oleic acid, linoleic acid, linolenic acid or isostearic acid, and mixtures thereof;
fatty acid esters, for example of 4 to 22 carbon atoms, and for example of octanoic acid, of heptanoic acid, of lanolic acid, of oleic acid, of lauric acid or of stearic acid, for instance propylene glycol dioctanoate, propylene glycol monoisostearate, polyglyceryl 2-diisostearate or neopentyl glycol dipheptanoate;
hydroxylated esters, for instance isostearyl lactate, octyl hydroxystearate, octyldodecyl hydroxystearate, diisostearyl malate, triisocetyl citrate, and glyceryl or diglyceryl triisostearate; diethylene glycol diisononanoate; and
pentaerythritol esters; esters of aromatic acids and of alcohols containing 4 to 22 carbon atoms, for exampletridecyl trimellitate,
fatty alcohols that are liquid at room temperature, with a branched and/or unsaturated carbon-based chain containing from 8 to 26 carbon atoms, for instance oleyl alcohol, linoleyl alcohol, linolenyl alcohol, isostearyl alcohol or octyldedecanol;
fluoro oils;
and mixtures thereof.

The non-volatile silicone oils that may be used in the composition according to the invention may be non-volatile polydimethylsiloxanes (PDMS), polydimethylsiloxanes comprising alkyl or alkoxy groups, which are pendent and/or at the end of a silicone chain, these groups each containing from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates, and mixtures thereof.

The non-volatile oils may be present in a composition according to the invention in a content ranging from 0.01% to 80% by weight, for example from 0.1% to 75% by weight and for example from 1% to 70% by weight relative to the total weight of the composition in water-in-oil emulsion form.

The volatile oils may be present in a composition according to the invention in a content ranging from 0.01% to 80% by weight, for example from 0.1% to 75% by weight and for example from 1% to 70% by weight relative to the total weight of the composition in water-in-oil emulsion form.

Solid Fatty Substances

The fatty phase of the composition according to the invention may comprise at least one fatty substance that is solid at room temperature and atmospheric pressure, and may be chosen from waxes, pasty fatty substances and gums, and mixtures thereof. This solid fatty substance may be present in a proportion of from 0.01% to 50%, for example from 0.1% to 25% and for example from 0.2% to 20% by weight relative to the total weight of the composition in the form of a water-in-oil emulsion.

Pasty Fatty Substances

Thus, the composition according to the invention may comprise at least one fatty compound that is pasty at room temperature.

For the purposes of the invention, the term “pasty fatty substance” means fatty substances with a melting point ranging from 20 to 55° C. and for example from 25 to 45° C., and/or a viscosity at 40° C. ranging from 0.1 to 40 Pa·s (1 to 400 poises) and for example from 0.5 to 25 Pa·s, measured using a Contraves TV or Rheomat 80 viscometer, equipped with a spindle rotating at 60 Hertz. A person skilled in the art can select the spindle for measuring the viscosity, from the spindles MS-r3 and MS-r4, on the basis of his general knowledge, so as to be able to perform the measurement of the pasty compound tested.

According to one exemplary embodiment, these fatty substances may be hydrocarbon-based compounds optionally of polymeric type; they may also be chosen from silicone compounds; they may also be in the form of a mixture of hydrocarbon-based and/or silicone compounds. In the case of a mixture of different pasty fatty substances, hydrocarbon-based pasty compounds (mainly containing carbon and hydrogen atoms and possibly ester groups) may for example be preferably used in major proportion.

Among the pasty compounds that may be used in the composition according to the invention, mention may be made for example lanolins and lanolin derivatives, for instance acetylated lanolins, oxypropylenated lanolins or isopropyl lanolate, with a viscosity of from 18 to 21 Pa·s and for example 19 to 20.5 Pa·s, and/or a melting point of 30 to 55° C., and mixtures thereof. Esters of fatty acids or of fatty alcohols, for example those containing 20 to 65 carbon atoms (melting point of about 20 to 35° C. and/or viscosity at 40° C. ranging from 0.1 to 40 Pa·s) may for example be used, for instance triisostearyl or cetyl citrate; arachidyl propionate; polyvinyl laurate; cholesterol esters, for instance triglycerides of plant origin such as hydrogenated plant oils, viscous polyesters, for instance poly(12-hydroxystearic acid), and mixtures thereof. Triglycerides of plant origin that may be used include hydrogenated castor oil derivatives, such as Thixinr from Rheox.

Mention may also be made of pasty silicone fatty substances such as for example high molecular weight polydimethylsiloxanes (PDMS) and for example those with pendent chains of the alkyl or alkoxy type containing from 8 to 24 carbon atoms, and a melting point of 20-55° C., for instance stearyl dimethicones, for example those sold by the company Dow Coming under the trade names DC2503® and DC25514®, and mixtures thereof.

The pasty fatty substance may be present in a composition according to the invention in a content ranging from 0.01% to 50% by weight, for example ranging from 0.1% to 45% by weight and for example ranging from 0.2% to 30% by weight relative to the total weight of the composition in the form of a water-in-oil emulsion.

Waxes

The fatty phase may also comprise a wax or a mixture of waxes. The wax may be solid at room temperature (25° C.), with a reversible solid/liquid change of state, having a melting point of greater than 30° C., which may be up to 120° C. It may be a hydrocarbon-based wax, a fluoro wax and/or a silicone wax and may be of animal, plant, mineral or synthetic origin.

Hydrocarbon-based waxes, for instance beeswax, lanolin wax, Chinese insect waxes; rice wax, carnauba wax, candelilla wax, ouricury wax, esparto grass wax, cork fibre wax, sugar cane wax, Japan wax and sumach wax; montan wax, microcrystalline waxes, paraffins and ozokerite; polyethylene waxes, the waxes obtained by Fisher-Tropsch synthesis and waxy copolymers, and also esters thereof and mixtures thereof, may for example be used.

The waxes obtained by catalytic hydrogenation of animal or plant oils containing linear or branched C₈-C₃₂ fatty chains may also be mentioned.

Among these waxes, mention may be made for example of hydrogenated jojoba oil, isomerised jojoba oil such as for example the partially hydrogenated trans-isomerised jojoba oil manufactured or sold by the company Desert Whale under the trade reference Iso-Jojoba-50®, hydrogenated sunflower oil, hydrogenated castor oil, hydrogenated coconut oil and hydrogenated lanolin oil, bis(l,1,1-trimethylolpropane) tetrastearate sold under the name Hest 2T-4S® by the company Heterene, and bis(l,1,l-trimethylolpropane) tetrabehenate sold under the name Hest 2T-4B® by the company Heterene.

Mention may also be made for example of silicone waxes and fluoro waxes.

The wax obtained by hydrogenation of olive oil esterified with stearyl alcohol, sold under the name Phytowax Olive 18 L57®, or the waxes obtained by hydrogenation of castor oil esterified with cetyl alcohol, sold under the name Phytowax Ricin 16L64® and 22L73® by the company Sophim, may also be used. Such waxes are described in patent application FR-A-2 792 190.

The wax may be present in a composition according to the invention in a content ranging from 0.01% to 50% by weight, for example from 0.1% to 30% by weight and for example from 0.2% to 20% by weight relative to the total weight of the composition in water-in-oil emulsion form.

Surfactants

The emulsion advantageously contains at least one surfactant that may be present for example in a proportion ranging from 0.1% to 30% by weight and for example from 5% to 15% by weight relative to the total weight of the composition in water-in-oil emulsion form.

The surfactant of these emulsions may for example be chosen from those of hydrocarbon-based, silicone and/or fluoro type.

The surfactant may be a single component or a mixture of surfactants; for example a mixture of surfactants chosen from those mentioned below.

These surfactants may be chosen from anionic and nonionic surfactants. Reference may be made to Kirk-Othmer's “Encyclopaedia of Chemical Technology”, volume 22, pp. 333-432, 3rd edition, 1979, Wiley, for the definition of the properties and functions of surfactants, for example pp. 347-377 of this reference, for the anionic and nonionic surfactants.

The surfactants that allow water-in-oil emulsions to be obtained are surfactants whose HLB (hydrophilic/lipophilic balance) is between 3 and 6. The definition of the HLB is given in the book Galenica 5, Les Systémes Dispersés-I Agents de Surface et Emulsions, F. Puisieux, M. Seiller, pages 153-155, published by Lavoisier.

According to one exemplary embodiment, the surfactants may be silicone surfactants.

The silicone surfactants that may be used in the cosmetic compositions in water-in-oil emulsion form according to the invention may be chosen for example from dimethicone copolyols, alkyldimethicone or alkoxydimethicone copolyols (for example cetyldimethicone copolyols), and mixtures thereof.

They may for example be selected from the group consisting of alkyl dimethicone copolyols and dimethicone copolyols.

The surfactants that may be used according to this exemplary embodiment of the invention, adapted to the production of a W/O emulsion, may be chosen from dimethicone copolyols such as for example the mixture of cyclomethicone and of dimethicone copolyol, for example sold under the name DC 5225 C by the company Dow Coming, and dimethicone copolyols such as for example lauryl methicone copolyol, for example sold under the name Dow Coming 5200 Formulation Aid® by the company Dow Coming and cetyldimethicone copolyol, for example sold under the name Abil EM 90R® by the company Goldschmidt, or polyglyceryl-4 isostearate/cetyldimethicone copolyol/hexyl laurate, for example sold under the name Abil WE 09® by the company Goldschmidt, and mixtures thereof.

Among these silicone surfactants, mention may also be made for example of those sold under the names Abil WS08® and Abil EM97® by the company Goldschmidt, DC-5200® and Q2-3225® by the company Dow Corning and 218-1138®, SF 1228® and SF1328® by the company General Electric.

According to another exemplary embodiment, the surfactants that are suitable for obtaining a water-in-oil W/O emulsion may be selected from the group comprising polyisobutylene surfactants with esterified succinic end groups, such as for example those sold under the names Lubrizol 5603® and Chemcinnate 2000® by the companies Lubrizol and Chemron (see FR 2 811 565 and WO 04/100 904).

According to another exemplary embodiment, the water-in-oil W/O emulsion surfactants may be of the emulsifying silicone elastomer type.

For the purposes of the invention, the term “emulsifying silicone elastomer” means a silicone elastomer comprising at least one hydrophilic chain, and which may be chosen from polyoxyalkylenated silicone elastomers.

The polyoxyalkylenated silicone elastomer is a crosslinked organopolysiloxane that may be obtained by addition-crosslinking reaction of a diorganopolysiloxane containing at least one hydrogen bonded to silicon and of a polyoxyalkylene containing at least two ethylenically unsaturated groups.

The polyoxyalkylenated silicone elastomer is generally conveyed in the form of a gel in at least one hydrocarbon-based oil and/or one silicone oil. Polyoxyalkylenated elastomers are for example described in patents U.S. Pat. Nos. 5,236,986, 5,412,004, 5,837,793 and 5,811,487, the content of which is incorporated by reference.

As polyoxyalkylenated silicone elastomers that are suitable for use in the present invention, mention may be made for example of those sold under the names KSG-21, KSG-20, KSG-30, KSG-31, KSG-32, KSG-33, KSG-210, KSG-310, KSG-320, KSG-330, KSG-340 and X-226146 by the company Shin-Etsu, or DC9010 and DC9011 by the company Dow Coming.

One or more co-emulsifiers, which may for example be chosen from the group comprising alkyl esters of polyols, may be added to the surfactants mentioned above. The alkyl ester of polyols may for example be a glycerol and/or sorbitan ester, for example selected from the group consisting of polyglyceryl isostearate, such as for example the product sold under the name Isolan GI 34® by the company Goldschmidt, sorbitan isostearate, such as the product sold under the name Arlacel 987® by the company ICI, sorbitan glyceryl isostearate, such as the product sold under the name Arlacel 986® by the company ICI, and mixtures thereof.

The surfactant and the co-emulsifier may for example be present in the composition in a total content ranging from 0.3% to 30% by weight and for example from 0.5% to 20% by weight relative to the total weight of the composition in water-in-oil emulsion form.

According to one exemplary embodiment, the water-in-oil emulsions according to the invention may be obtained according to a particular preparation process that consists in introducing the statistical copolymer with a linear main chain of ethylenic nature having a tensioning effect as defined above, after the preparation of the emulsion.

According to one exemplary embodiment, the statistical copolymer in accordance with the invention may be introduced into the composition with the aqueous phase, during the emulsification step.

Multiple Emulsions

According to another exemplary embodiment, the composition according to the invention may be in the form of a multiple emulsion, for example in the form of a water-in-oil-in-water, oil-in-water-in-oil or water-in-silicone oil-in-water emulsion.

According to one exemplary embodiment, the multiple emulsion is of the water-in-oil-in-water type.

As water-in-silicone oil-in-water emulsions that are suitable for use in the present invention, mention may be made, for example, of those described in patent EP 0 648 102.

According to one exemplary embodiment, the outer aqueous phase of the water-in-silicone oil-in-water emulsion may be gelled.

In the case of a water-in-oil-in-water (W/O/W) multiple emulsion, the composition according to the invention may be prepared according to any method known to those skilled in the art.

It may for example be an emulsification of a water-in-oil (W/O) emulsion, referred to as the primary emulsion, in a gelled or non-gelled aqueous phase, referred to as the outer aqueous phase.

According to one exemplary embodiment, the multiple emulsion may be obtained by emulsifying a water-in-oil emulsion is a gelled aqueous phase.

The primary emulsion may in particular be in accordance with a water-in-oil (W/O) emulsion as described above.

Outer Aqueous Phase

To ensure the emulsification of the primary emulsion in the continuous outer aqueous phase of the emulsion, it may contain, according to a first variant, at least one surfactant copolymer consisting of a major fraction of a monoolefinically unsaturated C₃-C₆ carboxylic acid monomer or the anhydride thereof and a minor fraction of an acrylic acid fatty ester monomer. Such copolymers are described in patent application EP 0 268 164 and are obtained according to the preparation methods described in the said document. It is possible, for example, to use a fatty-chain polymer of the C₃-C₆ monoethylenic carboxylic acid or anhydride/fatty-chain acrylic ester copolymer type.

This fatty-chain polymer may be chosen for example from the copolymers sold under the names Pemulen® and Carbopol 1342® or Carbopol 1382® by the company Goodrich.

In one exemplary embodiment, it may for example be an acrylate/C₁₀-C₃₀-alkylacrylate copolymer, and for example the product sold under the name Pemulen TR1® or Pemulen TR2® by the company Goodrich.

This type of triple emulsion is for example described in patent applications EP0 908 170and EP0 648 102.

In a W/O/W multiple emulsion, the inner water dispersed in the fatty phase is separated from the outer water by an oil membrane. To ensure the stability of the system, it may be necessary to gel one or the other part in order to avoid leakage of the inner water towards the outer water.

Hydrophilic gelling agents that may be mentioned include for example carboxyvinyl polymers (carbomer), acrylic copolymers such as acrylate/alkylacrylate copolymers, polyglyceryl methacrylates, polyacrylamides, polysaccharides, natural gums and clays, and lipophilic gelling agents that may be mentioned include modified clays, for instance bentones, metal salts of fatty acids, hydrophobic silica and polyethylenes.

Examples of carboxyvinyl polymers that may be mentioned include those sold under the names Carbopol 980®, Carbopol 942® and Carbopol 950® by the company Goodrich or the product sold under the name Synthalen K® by the company Sigma.

According to another exemplary embodiment, a combination of two gelling agents may be used in the outer aqueous phase, consisting on the one hand of an alkali metal salt of an acrylic copolymer sold under the name Hostacerin PN 73® by the company Hoechst, and on the other hand of a polyglyceryl methacrylate manufactured under the name Lubragel MS® by the company United Guardian Inc.

According to a second variant, the continuous outer aqueous phase of the emulsion may contain as gelling agent a polymer or copolymer of acrylic or methacrylic acid combined with a polyglyceryl methacrylate, and the fatty phase may contain a fluoro oil. This type of triple emulsion is for example described in patent application EP 0 507 693.The gelled aqueous second phase of the compositions according to this second variant may for example comprise a surfactant chosen for example from sucrose esters.

According to a third variant, one of the aqueous phases of the water-in-oil-in-water W/O/W emulsion may have a water activity value of less than or equal to 0.85.

The water activity a_{w} of a medium containing water is the ratio of the vapour pressure of water of the medium “P_{H20} medium” to the vapour pressure of pure water “P_{H20} pure” at the same temperature. It may also be expressed as the ratio of the number of water molecules “N_{H20} to the total number of molecules N_{H20}+N_{dissolved substance}” which takes into account those of the dissolved substances “N_{dissolved substance}”.

It is given by the following formulae:

$${a}_{w} = \frac{{P}_{H2 \; 0 medium}}{{P}_{H 20 pure}} = \frac{{N}_{H 20}}{{N}_{H 20} + {N}_{dissolved substance}}$$

Various methods may be used to measure the water activity. The most common is the manometric method via which the vapour pressure is measured directly.

A cosmetic or dermatological composition conventionally has a water activity of about 0.95 to 0.99. A water activity of less than 0.85 represents an appreciable decrease in water activity.

A water activity value of less than or equal to 0.85 may especially be obtained by incorporation of an effective amount of polyol as described in patent application EP 0 779 071. To have such a water activity of less than 0.85, the amount of polyol in the phase under consideration, for example the continuous outer aqueous phase, should range from 35% to 90% by weight and for example from 60% to 85% by weight relative to the total weight of the aqueous phase with a low water activity, i.e. greater than 30% by weight relative to the total weight of the emulsion and for example ranging from 35% to 70% by weight relative to the total weight of the emulsion. As polyols that may be used to prepare such emulsions, mention may be made for example of glycerols and glycols, for example propylene glycol and polyethylene glycols. According to one exemplary embodiment, the polyol(s) is (are) totally or partially in a form complexed with an acrylic or methacrylic polymer.

The content of surfactant copolymer as described above in the emulsion according to the invention may range from 0.05% to 3% by weight and for example from 0.1% to 2% by weight relative to the total weight of the multiple emulsion.

The primary emulsion may be prepared according to the process that consists in introducing into the emulsion the statistical copolymer with a tensioning effect according to the invention in the form of an aqueous dispersion, with the inner aqueous phase.

The composition of the invention may for example constitute an anti-ageing and for example anti-wrinkle composition. However, according to one exemplary embodiment, it may constitute a bodycare composition and for example a slimming composition.

Adjuvants

In a known manner, the composition of the invention may also contain adjuvants that are common in cosmetics, such as hydrophilic or lipophilic gelling agents, hydrophilic or lipophilic active agents, preserving agents, antioxidants, solvents, fragrances, fillers, film-forming polymers, screening agents, pigments, odour absorbers and dyestuffs. The amounts of these various adjuvants are those conventionally used in the field under consideration, for example from 0.01% to 20% of the total weight of the composition. Depending on their nature, these adjuvants may be introduced into the fatty phase, into the aqueous phase, into lipid vesicles and/or into nanoparticles. These adjuvants and the concentrations thereof should be such that they do not modify the desired tensioning property of the statistical copolymer with a linear main chain of ethylenic nature.

In addition, other compounds known to those skilled in the art as tensioning agents and having properties different from those of the agents used according to the invention may also be combined with the tensioning agents used according to the invention, for example synthetic latices, plant proteins, polysaccharides of plant origin optionally in the form of microgels, starches, mixed silicates and colloidal particles of mineral fillers.

As a variant or in addition, when the composition according to the invention is a slimming composition, it may for example comprise, in a physiologically acceptable medium, at least one statistical copolymer with a linear main chain of ethylenic nature as defined above and one or more draining, lipolytic, de-infiltrating, slimming, firming, anti-glycating and/or vaso-protective compounds.

The amount of slimming active agent(s) may vary within a wide range and depends on the nature of the active agent(s) used. In general, the slimming active agent(s) is (are) present in a concentration ranging from 0.05% to 20% and for example from 0.1% to 10% by weight relative to the total weight of the composition.

When the composition according to the invention constitutes an anti-ageing composition, at least one compound chosen from: desquamating agents; moisturizers; depigrnenting or pro-pigmenting agents; anti-glycation agents; NO-synthase inhibitors; agents for stimulating the synthesis of dermal or epidermal macromolecules and/or for preventing their degradation; agents for stimulating the proliferation of fibroblasts and/or keratinocytes or for stimulating keratinocyte differentiation; other muscle relaxants and/or dermo-decontracting agents; tensioning agents; anti-pollution agents and/or free-radical scavengers; agents acting on the capillary circulation; agents acting on the energy metabolism of cells; and mixtures thereof, may be introduced in the composition.

Examples of such additional compounds are: retinol and its derivatives such as retinyl palmitate; ascorbic acid and its derivatives such as magnesium ascorbyl phosphate and ascorbyl glucoside; tocopherol and its derivatives such as tocopheryl acetate; nicotinic acid and its precursors such as nicotinamide; ubiquinone; glutathione and its precursors such as L-2-oxothiazolidine-4-carboxylic acid; plant extracts and especially plant proteins and hydrolysates thereof, and also plant hormones; marine extracts such as algal extracts; bacterial extracts; sapogenins such as diosgenin and wild yam extracts containing them; ceramides; hydroxy acids, such as salicylic acid and 5-n-octanoylsalicylic acid; resveratrol; oligopeptides and pseudodipeptides and acyl derivatives thereof; manganese nd magnesium salts, for example the gluconates; and mixtures thereof.

The amounts of these active agents may vary within a wide range. In general, these active agents are present in a concentration ranging from 0.01% to 15% and for example from 0.05% to 10% by weight relative to the total weight of the composition.

In the event of incompatibility, the active agents indicated above may be incorporated into spherules, for example ionic or nonionic vesicles and/or nanoparticles (nanocapsules and/or nanospheres), so as to isolate them from each other in the composition.

The composition according to the invention may also be combined with another cosmetic composition.

Thus, according to one exemplary embodiment the present invention also relates to a cosmetic product comprising at least:
one first composition, in the form of a water-in-oil emulsion or a multiple emulsion according to the invention, containing at least one statistical copolymer with a linear main chain of ethylenic nature as defined above, and
one second composition comprising at least one physiologically acceptable medium.

The first composition of the product according to the invention may for example constitute a base coat applied to the keratin material, and the second composition a topcoat. However, it is possible to apply onto the second coat an overcoat that may or may not have the constitution of the second coat.

The product according to the invention may comprise two (or more) physiologically acceptable compositions packaged separately or together in the same packaging article or in two (or more) separate or distinct packaging articles.

According to another exemplary embodiment, the present invention relates to a kit of compositions comprising a product according to the invention.

These compositions may be for example packaged separately and for example in separate or distinct compartments or containers.

According to one exemplary embodiment, these compositions may be packaged in the form of a double-pump distribution assembly, for instance those described in patents U.S. Pat Nos. 5,692,644, 5,884,759, 5,875,888, 5,875,889, 5,992,693, 5,944,175 and 6,402,364.

The examples below of compositions according to the invention are given as illustrations and with no limiting nature. The amounts therein are given as weight percentages. The degrees of neutralization of the copolymers are 100%.

In the examples that follow, “Mp” is the abbreviation for the peak molecular mass; “Mn” for the number-average molecular mass, “Mw” for the weight-average molecular mass and “Ip” for the polydispersity index.

### EXAMPLES

### Example 1

Example of Synthesis of a Methyl Methacrylate/Methacrylic Acid Statistical Copolymer

1st step: Synthesis of the polymer

1 g of Trigonox 21S (t-butylperoxy 2-ethylhexanoate) and 200 g of methyl ethyl ketone are placed in a 21 jacketed reactor. The mixture is refluxed for one hour. After one hour, a mixture of 170 g of methyl methacrylate and 30 g of methacrylic acid is added dropwise over a period of one hour. The colourless mixture becomes viscous. Heating is stopped six hours after addition of the monomers.

Composition by NMR: 85.1% methyl methacrylate, 14.9% methacrylic acid

Mass by GPC in tetrahydrofuran (THF) (polystyrene standards): Mp=98,772 g.mol⁻¹; Mn=61,261 g.mol⁻¹; Mw=105,698 g.mol⁻¹ Ip=1.7

2nd step: Dispersion of the polymer in water

200 g of methyl ethyl ketone are added to the above reaction medium and the mixture is heated to 60° C. 30.86 g of 2-amino-2-methylpropanol are added dropwise to neutralize the acid functions, and 1200 g of water are added. The volatile solvents are evaporated off by heating to 100° C. A transparent yellow aqueous dispersion is obtained.

The glass transition temperature of this copolymer, determined by DSC according to the protocol given in the description, is 81° C.

### Example 2

Cosmetic Composition in the Form of a W/O/W Emulsion

The composition below was prepared:

| | |
|---|---|
| Primary emulsion (A): | |
| Water | 10.20 g |
| 40/30/30 mixture of cetyl dimethicone copolyol, | 3.50 g |
| polyglyceryl-4 isostearate and hexyl laurate (Abil ® | |
| WE 09 from Goldschmidt) | |
| Cyclopentasiloxane | 16.50 g |
| Dimethicone | 4.00 g |
| Ethylenic copolymer according to the invention (Example 1) | 65.00 g |
| Magnesium sulfate | 0.80 g |
| Multiple emulsion: | |
| Primary emulsion (A): | 22.50 g |
| Cyclopentasiloxane | 3.50 g |
| Apricot kernel oil | 4.00 g |
| Water | 68.05 g |
| Preserving agents | 1.00 g |
| Pentasodium ethylenediaminetetramethylenephosphonate | 0.05 g |
| Acrylic acid/stearyl methacrylate copolymer (Pemulen TR1 ® | 0.60 g |
| (Novéon)) | |
| Sodium hydroxide | 0.30 g |

Procedure

Preparation of the Primary Emulsion:

The Abil® WE 09, the cyclopentasiloxane and the dimethicone are homogenized at room temperature with stirring. The water and the copolymer according to Example 1 in accordance with the invention are incorporated slowly with vigorous stirring.

Preparation of the Triple Emulsion:

The alkyl acrylate copolymer, the preserving agents and the sequestrant (pentasodium ethylenediaminetetramethylenephosphonate) are dispersed at room temperature with stirring. The mixture is left to swell for about 45 minutes with stirring and is then neutralized with sodium hydroxide. The primary emulsion is diluted with the cyclopentasiloxane and the apricot kernel oil and this mixture is then incorporated slowly into the aqueous phase with stirring.

### Example 3

Cosmetic Composition in the Form of a W/O Emulsion

The composition below was prepared:

| | |
|---|---|
| A | |
| Cetyl dimethicone copolyol (Abil ® EM 90 from | 1.50 g |
| the company Goldschmidt) | |
| Polyglyceryl isostearate | 0.50 g |
| Isohexadecane | 4.00 g |
| Squalane | 1.85 g |
| Dimethicone | 2.05 g |
| Apricot kernel oil | 1.10 g |
| Cyclopentasiloxane | 9.00 g |
| Propylparaben | 0.15 g |
| B | |
| Water | 30.70 g |
| Propylene glycol | 3.00 g |
| Magnesium sulfate | 1.75 g |
| Methylparaben | 0.20 g |
| Preserving agent | 0.30 g |
| C | |
| Ethylenic copolymer according to the invention (Example 1) | 40.90 g |
| D | |
| Nylon 12 | 3.00 g |

Procedure:

Phase A and phase B are homogenized separately at room temperature with stirring.

The emulsion is prepared by incorporating phase B into phase A.

Phases C and D are incorporated with stirring.

Results:

Stability test

The stability after two months of storage at 4° C., 25° C., 37° C. and 45° C. of the compositions of Examples 2 and 3 above (containing, respectively, 7% and 2.5% by weight, as active material, of tensioning copolymer in accordance with the invention) was studied. The results are collated in the table below:

| Composition | Stability |
|---|---|
| Example 2: W/O/W emulsion containing 2.5%, as active | yes |
| material, of copolymer according to Example 1. | |
| Example 3: W/O emulsion containing 7%, as active material, of | yes |
| copolymer according to Example 1. | |

It emerges from the above table that the compositions according to the invention (Examples 2 and 3) are stable, this being the case at all the storage temperatures.

1. Cosmetic composition comprising, in a physiologically acceptable medium, at least one statistical copolymer with a linear main chain of ethylenic nature, in which the said copolymer:
(i) has a weight-average molecular mass of between 15,000 and 600,000 g/mol,
(ii) contains at least 70% of monomer units derived from monomers whose homopolymers are hydrophobic and have a glass transition temperature of greater than 40° C.,
(iii) also contains at least one “monomer unit” derived from an at least partially neutralized ionic hydrophilic monomer, and
(iv) has an overall glass transition temperature of greater than or equal to 45° C., and being in the form of a water-in-oil emulsion or a multiple emulsion.

2. Composition according to claim 1, in which the copolymer comprises more than 75% by weight, relative to its total weight, of “monomer units” derived from monomers whose homopolymers have a glass transition temperature of greater than 400° C.

3. Composition according to claim 1, in which the copolymer comprises more than 80% by weight, relative to its total weight, of “monomer units” derived from monomers whose homopolymers have a glass transition temperature of greater than 40° C.

4. Composition according to claim 1, in which the copolymer comprises up to 95% by weight, relative to its total weight, of “monomer units” derived from monomers whose homopolymers have a glass transition temperature of greater than 40° C.

5. Composition according to claim 1, in which the monomers whose homopolymers have a glass transition temperature of greater than 40° C. are chosen from vinyl compounds, acrylates, methacrylates and (meth)acrylamides.

6. Composition according to claim 1, in which the monomers whose homopolymers have a glass transition temperature of greater than 40° C. are chosen from:
the vinyl compounds of formula:
CH₂═CHR₁,
in which R₁ is a group 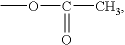
a C₃ to C₈ cycloalkyl group or a C₆ to C₂₀ aryl group,
the acrylates of formula:
CH₂═CHCOOR₂
in which R₂ is a tert-butyl group, a C₃ to C₈ cycloalkyl group optionally bridged with a C₁ to C₄ alkylene group, or a C₇ to C₃₀ aralkyl group having a C₁ to C₄ alkyl group,
the methacrylates of formula:
CH₂═C(CH₃)COOR₃
in which R₃ is an isobutyl or tert-butyl group, a linear or branched C₁ to C₃ alkyl group, a C₃ to C₈ cycloalkyl group optionally bridged with a C₁ to C₄ alkylene group, or a C₇ to C₃₀ aralkyl group having a C₁ to C₄ alkyl group, and
the (meth)acrylamides of formula: 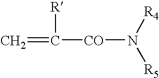
in which R′ denotes H or —CH₃, and in which R₄ and R₅, which may be identical or different, each represent a hydrogen atom or a linear or branched C₄ to C₁₂ alkyl group, it being understood that R₄ and R₅ cannot simultaneously represent a hydrogen atom.

7. Composition according to claim 1, in which the monomers whose homopolymers have a glass transition temperature of greater than 40° C. are selected from the group consisting of styrene, benzyl acrylate, C₃ to C₈ cycloalkyl acrylate optionally bridged with a C₁ to C₄ alkylene group, tert-butyl acrylate, C₁ to -C₃ alkyl methacrylate, tert-butyl methacrylate, benzyl methacrylate and C₃ to C₈ cycloalkyl methacrylate optionally bridged with a C₁ to C₄ alkylene group.

8. Composition according to claim 1, in which the monomers whose homopolymers have a glass transition temperature of greater than 40° C. are selected from the group consisting of vinylcyclohexane, styrene, vinyl acetate, benzyl acrylate, cyclohexyl acrylate, tert-butyl acrylate, isobomyl acrylate, norbomyl acrylate, methyl, ethyl, isobutyl, cyclohexyl, benzyl, tert-butyl, isobomyl or norbornyl methacrylate, N-butylacrylamide, N-t-butylacrylamide and N,N-dibutylacrylamide.

9. Composition according to claim 1, in which the monomers whose homopolymers have a glass transition temperature of greater than 40° C. are selected from the group consisting of benzyl acrylate, cyclohexyl acrylate, tert-butyl acrylate, isobornyl acrylate, norbornyl acrylate, methyl, ethyl, isobutyl, cyclohexyl, benzyl, tert-butyl, isobornyl or norbornyl methacrylate and styrene.

10. Composition according to claim 1, in which the monomers whose homopolymers have a glass transition temperature of greater than 40° C. are selected from the group consisting of methyl methacrylate and cyclohexyl methacrylate.

11. Composition according to claim 1, in which the ionic hydrophilic monomer is selected from the group consisting of anionic hydrophilic monomers, cationic hydrophilic monomers and amphoteric monomers, and mixtures thereof.

12. Composition according to claim 11, in which the anionic hydrophilic monomer is defined by formula (I):
CH₂═CR₆(Z)ₙ(R₇)ₘY (I)
in which —Z has one of the following meanings: C(═O)O, C(═O)NH, O, O(C═O),
n is equal to 0 or 1,
m is equal to 0 or 1,
R₆ is a hydrogen atom, a CH₃ group or a (C₂-C₃₀)alkyl group,
R₇ is selected from the group consisting of linear, saturated or unsaturated and/or branched and/or cyclic (aromatic or non-aromatic) C₁ to C₃₀ alkylene groups, optionally including one or more heteroatoms, and
Y is selected from the following groups: —COOH, —SO₃H, —OSO₃H, —OP(OH)₂ and —OPO(OH)₂.

13. Composition according to claim 12, in which R₆ is a hydrogen atom or a CH₃ or C₂H₅ group and R₇ is selected from the group consisting of C₁ to C₃₀ alkylene, phenylene, benzylene, —(CH₂-CH═CH)— and (CHOH) groups.

14. Composition according to claim 11, in which the anionic hydrophilic monomer is chosen from:
ethylenically unsaturated monomers comprising at least one carboxylic acid (COOH), phosphonic acid (PO₃H₂) or sulfonic acid (SO₃H) function,
carboxylic anhydrides bearing a vinyl bond,
diacids,
and mixtures thereof.

15. Composition according to claim 1, in which the hydrophilic monomer is (meth)acrylic acid.

16. Composition according to of claim 1, in which the weight content of “monomer units” derived from ionic hydrophilic monomers in the copolymer is between 5% and 30% by weight relative to the total weight of the copolymer.

17. Composition according to claim 1, in which the weight content of “monomer units” derived from ionic hydrophilic monomers in the copolymer is between 10% and 25% by weight relative to the total weight of the copolymer.

18. Composition according to claim 1, in which the weight content of “monomer units” derived from ionic hydrophilic monomers in the copolymer is between 10% and 20% by weight relative to the total weight of the copolymer.

19. Composition according to claim 1, in which the copolymer is selected from the group consisting of:
methyl methacrylate/methacrylic acid copolymers; methyl methacrylate/acrylic acid copolymers, the said copolymers containing between 70% and 90% by weight of methyl methacrylate;
ethyl methacrylate/methacrylic acid copolymers; ethyl methacrylate/acrylic acid copolymers, the said copolymers containing between 70% and 90% by weight of ethyl methacrylate;
isobutyl methacrylate/methacrylic acid copolymers; isobutyl methacrylate/acrylic acid copolymers, the said copolymers containing between 70% and 90% by weight of isobutyl methacrylate;
benzyl methacrylate/methacrylic acid copolymers; benzyl methacrylate/acrylic acid copolymers, the said copolymers containing between 70% and 90% by weight of benzyl methacrylate;
benzyl acrylate/methacrylic acid copolymers; benzyl acrylate/acrylic acid copolymers, the said copolymers containing between 70% and 90% by weight of benzyl acrylate;
cyclohexyl methacrylate/methacrylic acid copolymers; cyclohexyl methacrylate/acrylic acid copolymers, the said copolymers containing between 70% and 90% by weight of cyclohexyl methacrylate;
cyclohexyl acrylate/methacrylic acid copolymers; cyclohexyl acrylate/acrylic acid copolymers, the said copolymers containing between 70% and 90% by weight of cyclohexyl acrylate;
tert-butyl methacrylate/methacrylic acid copolymers; tert-butyl methacrylate/acrylic acid copolymers, the said copolymers containing between 70% and 90% by weight of tert-butyl methacrylate;
tert-butyl acrylate/methacrylic acid copolymers; tert-butyl acrylate/acrylic acid copolymers, the said copolymers containing between 70% and 90% by weight of tert-butyl acrylate;
isobomyl methacrylate/methacrylic acid copolymers; isobomyl methacrylate/acrylic acid copolymers, the said copolymers containing between 70% and 90% by weight of isobomyl methacrylate;
isobomyl acrylate/methacrylic acid copolymers; isobomyl acrylate/acrylic acid copolymers, the said copolymers containing between 70% and 90% by weight of isobomyl acrylate;
norbomyl methacrylate/methacrylic acid copolymers; norbomyl methacrylate/acrylic acid copolymers, the said copolymers containing between 70% and 90% by weight of norbornyl methacrylate;
norbomyl acrylate/methacrylic acid copolymers; norbornyl acrylate/acrylic acid copolymers, the said copolymers containing between 70% and 90% by weight of norbornyl acrylate; and
styrene/methacrylic acid copolymers; styrene/acrylic acid copolymers, the said copolymers containing between 70% and 90% by weight of styrene.

20. Composition according to claim 1, in which the copolymer also comprises at least one “monomer unit” derived from a monomer whose homopolymer has a glass transition temperature of less than 40° C.

21. Composition according to claim 20, in which the monomer whose homopolymer has a glass transition temperature of less than 40° C. is present in a content of less than or equal to 25% by weight, relative to the total weight of the copolymer.

22. Composition according to claim 20, in which the monomer whose homopolymer has a glass transition temperature of less than 40° C. is present in a content ranging from 5% to 25% by weight relative to the total weight of the copolymer.

23. Composition according to claim 20, in which the monomer whose homopolymer has a glass transition temperature of less than 40° C. is present in a content of less than or equal to 10% by weight relative to the total weight of the copolymer.

24. Composition according to claim 20, in which the monomer whose homopolymer has a glass transition temperature of less than 40° C. is present in a content ranging from 5% to 10% by weight relative to the total weight of the copolymer.

25. Composition according to claim 1, in which the copolymer is included in a content, expressed as dry matter, ranging from 0.1% to 15% by weight relative to the total weight of the composition.

26. Composition according to claim 1, in which the copolymer is included in a content, expressed as dry matter, ranging from 1% to 10% by weight relative to the total weight of the composition.

27. Composition according to claim 1, in which the copolymer is included in a content, expressed as dry matter, ranging from 1% to 5% by weight relative to the total weight of the composition.

28. Composition according to claim 14, in which the ionic hydrophilic monomer includes an anionic hydrophilic monomer selected from the group consisting of acrylic acid, methacrylic acid, 2-carboxyethyl (meth)acrylate, vinylbenzoic acid, acrylamidoglycolic acid CH₂═CHCONHCH(OH)CO₂H, maleic acid, acrylamidopropanesulfonic acid, acrylamido-2-methylpropanesulfonic acid, styrenesulfonic acid, vinylsulfonic acid, 3-propylsulfonic acid methacrylate or acrylate (CH₂═C(CH₃)CO₂(CH₂)₃SO₃H), 2-ethylsulfonic acid methacrylate or acrylate (CH₂═C(CH₃)CO₂(CH₂)₂SO₃H) and methyl vinyl sulfone, vinylphosphonic acid (CH₂═CH—PO(OH)₂) and 2-ethylphosphonic acid methacrylate (CH₂═C(CH₃)COOCH₂CH₂OP(O)(OH)₂), maleic anhydride, crotonic acid, itaconic acid, fumaric acid and maleic acid and mixtures thereof.

29. Composition according to claim 1, which is in the form of a water-in-oil emulsion.

30. Composition according to claim 29, in which the surfactant is selected from the group consisting of silicone surfactants, polyisobutylene surfactants with esterified succinic end groups and emulsifying silicone elastomers, and mixtures thereof.

31. Composition according to claim 29, in which the inner aqueous phase is non gelled.

32. Composition according to claim 30, in which the said silicone surfactant is selected from the group consisting of alkyl dimethicone copolyols and dimethicone copolyols.

33. Composition according to claim 30, in which the silicone surfactant is chosen from the mixture of cyclomethicone and of dimethicone copolyol, lauryl methicone copolyol, cetyl dimethicone copolyol, and the polyglyceryl-4 isostearate/cetyldimethicone copolyol/hexyl laurate, and mixtures thereof.

34. Composition according to claim 29, in which the fatty phase comprises at least one silicone oil.

35. Composition according to claim 29, also comprising at least one co-emulsifier.

36. Composition according to claim 35, in which the said co-emulsifier is a alkyl ester of polyols.

37. Composition according to claim 36, in which the alkyl ester of polyols is a glycerol and/or sorbitan ester selected from the group consisting of polyglyceryl isostearate, sorbitan isostearate and sorbitan glyceryl isostearate, and mixtures thereof.

38. Composition according to claim 1, which is in the form of a water-in-oil-in-water multiple emulsion.

39. Composition according to claim 38, which is in the form of a multiple emulsion obtained by emulsifying a water-in-oil emulsion comprising an inner aqueous phase and a fatty phase comprising at least one oil and at least one surfactant, in a gelled aqueous phase.

40. Composition according to claim 39, in which the outer aqueous phase contains at least one surfactant copolymer consisting of a major fraction of a monoolefinically unsaturated C₃C₆ carboxylic acid monomer or the anhydride thereof and of a minor fraction of acrylic acid fatty ester monomer.

41. Composition according to claim 40, in which the surfactant copolymer is an acrylate/C₁₀-C₃₀ alkyl acrylate copolymer.

42. Composition according to claim 39, in which one of the aqueous phases has a water activity value of less than or equal to 0.85.

43. Cosmetic method for tensioning the skin comprising applying to said skin a cosmetic composition in the form of a water-in-oil emulsion or a multiple emulsion containing a polymer as described in claim 1.

44. Cosmetic method for reducing or treating age signs, comprising applying to the skin at least one composition comprising, in a physiologically acceptable medium, at least one statistical copolymer with a linear main chain of ethylenic nature, in which the said copolymer:
(i) has a weight-average molecular mass of between 15,000 and 600,000 g/mol,
(ii) contains at least 70% of monomer units derived from monomers whose homopolymers have a glass transition temperature of greater than 40° C.,
(iii) also contains at least one “monomer unit” derived from an at least partially neutralized ionic hydrophilic monomer, and
(iv) has an overall glass transition temperature of greater than or equal to 45° C.,
and being in the form of a water-in-oil emulsion or a multiple emulsion, in an amount that is effective for smoothing out or effacing wrinkles and fine lines on human skin by means of a tensioning effect.

45. Cosmetic product comprising at least (i) one first composition in the form of a water-in-oil emulsion or a multiple emulsion, containing at least one copolymer as described in claim 1, and (ii) one second composition comprising at least one physiologically acceptable medium.

46. Kit of compositions comprising a product according to claim 45.

47. Kit according to claim 46, in which the first and second compositions are packaged in separate compartments or containers.

48. Kit according to claim 47, in which the said compositions are packaged in the form of a double-pump distribution assembly.
